(19) United States
(12) Patent Application Publication
(10) Pub. No.: US 2018/0363026 A1
(43) Pub. Date: Dec. 20, 2018

(54) STABILIZATION OF PCR REAGENTS AND ASSAYS

(71) Applicant: Biomatrica, Inc., San Diego, CA (US)

(72) Inventors: Joel DESHARNAIS, San Diego, CA (US); Colin POWERS, San Diego, CA (US)

(21) Appl. No.: 15/780,580

(22) PCT Filed: Dec. 6, 2016
(86) PCT No.: PCT/US16/65200

§ 371 Date: May 31, 2018

Related U.S. Application Data
(60) Provisional application No. 62/264,758, filed on Dec. 8, 2015.

(51) Int. Cl. C12Q 1/6806

(52) U.S. Cl. CPC C12Q 1/6806

(57) ABSTRACT

The present invention relates to stabilization of one or more component of a PCR or RT-PCR reaction mixture at ambient temperatures. In particular, formulations, compositions, articles of manufacture, kits and methods for substantially stable storage of one or more component of a PCR or RT-PCR reaction mixture at ambient temperatures are provided.

### CROSS-REFERENCE

This application claims the benefit of US Provisional Application No. 62/264,758, filed Dec. 8, 2015, which is incorporated by reference herein in its entirety.

### BACKGROUND OF THE INVENTION

There exists a need for improved formulations for and methods of stabilizing one or more component of a PCR or RT-PCR reaction mixture at ambient temperatures for a time sufficient for storage and shipping of the one or more component of a PCR or RT-PCR reaction mixture for research, diagnostic and therapeutic purposes.

### SUMMARY OF THE INVENTION

Described herein, in some embodiments, are formulations for substantially stable storage of one or more component of a PCR or RT-PCR reaction mixture at ambient temperatures, comprising: (i) a hydroxyacid; and (ii) a borate composition, wherein the one or more component of a PCR or RT-PCR reaction mixture remains stabilized after storage at ambient temperatures for a period of at least three months. In some embodiments, the hydroxyacid is selected from the group consisting of a compound of formula (I):

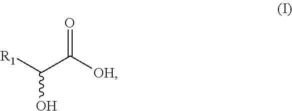

wherein R1 is selected from H, unsubstituted or substituted alkyl, and unsubstituted or substituted aryl. In some embodiments, the hydroxyacid is selected from the group consisting of lactic acid, malic acid, malonic acid, tartaric acid, citric acid, and a combination thereof. In some embodiments, the hydroxyacid is lactic acid. In some embodiments, the hydroxyacid is other than malic acid, malonic acid, tartaric acid, or citric acid. In some embodiments, the hydroxyacid is not malic acid. In some embodiments, the hydroxyacid is not malonic acid. In some embodiments, the hydroxyacid is not tartaric acid. In some embodiments, the hydroxyacid is not citric acid. In some embodiments, the borate composition is selected from the group consisting of boric acid, borate, sodium tetraborate, borax, and a combination thereof. In some embodiments, the borate composition is selected from the group consisting of boric acid, sodium tetraborate, and a combination thereof. In some embodiments, the formulation comprises lactic acid and boric acid. In some embodiments, the formulation comprises lactic acid, boric acid, and sodium tetraborate. In some embodiments, the formulation is selected from the formulations of Table 2.

Described herein, in some embodiments, are formulations for substantially stable storage of one or more component of a PCR or RT-PCR reaction mixture at ambient temperatures, comprising: (i) a non-reducing sugar; and (ii) a buffer, wherein the one or more component of a PCR or RT-PCR reaction mixture remains stabilized after storage at ambient temperatures for at least three months. In some embodiments, the non-reducing sugar is selected from the group consisting of sucrose and trehalose. In some embodiments, the non-reducing sugar is sucrose. In some embodiments, the buffer is selected from the group consisting of Tris-HCl, citric acid, tartaric acid, malic acid, sulfosalicylic acid, sulfoisophthalic acid, oxalic acid, borate, CAPS (3-(cyclohexylamino)-1-propanesulfonic acid), CAPSO (3-(cyclohexylamino)-2-hydroxy-1-propanesulfonic acid), EPPS (4-(2-hydroxyethyl)-1-piperazinepropanesulfonic acid), HEPES (4-(2-hydroxyethyl)piperazine-1-ethanesulfonic acid), MES (2-(N-morpholino)ethanesulfonic acid), MOPS (3-(N-morpholino)propanesulfonic acid), MOPSO (3-morpholino-2-hydroxypropanesulfonic acid), PIPES (1,4-piperazinediethanesulfonic acid), TAPS (N-[tris(hydroxymethyl)methyl]-3-aminopropanesulfonic acid), TAPSO (2-hydroxy-3-[tris(hydroxymethyl)methylamino]-1-propanesulfonic acid), TES (N-[tris(hydroxymethyl)methyl]-2-aminoethanesulfonic acid), bicine (N,N-bis(2-hydroxyethyl)glycine), tricine (N-[tris(hydroxymethyl)methyl]glycine), tris (tris(hydroxymethyl)aminomethane), and bis-tris (2-[bis(2-hydroxyethyl)amino]-2-(hydroxymethyl)-1,3-propanediol). In some embodiments, the formulation comprises Tris-HCl. In some embodiments, the formulation comprises sucrose and Tris-HCl. In some embodiments, the formulation is selected from formulations 12 or 13 of Table 3.

Described herein, in some embodiments, are formulations for substantially stable storage of one or more component of a PCR or RT-PCR reaction mixture at ambient temperatures, comprising: (i) an aminosulfonic acid or ammonium sulfate; and (ii) at least one of a non-reducing sugar, an amide, or polyethylene glycol, wherein the one or more component of a PCR or RT-PCR reaction mixture remains stabilized after storage at ambient temperatures for a period of at least three months. In some embodiments, the formulation comprises an aminosulfonic acid of formula (II):

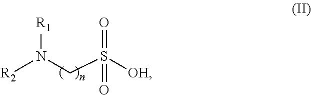

wherein R1 and R2 are independently selected from H, unsubstituted or substituted alkyl, and unsubstituted or substituted aryl. In some embodiments, the aminosulfonic acid is taurine. In some embodiments, the formulation comprises ammonium sulfate. In some embodiments, the formulation comprises a non-reducing sugar selected from the group consisting of sucrose and trehalose. In some embodiments, the non-reducing sugar is sucrose. In some embodiments, the formulation comprises an amide of formula (III):

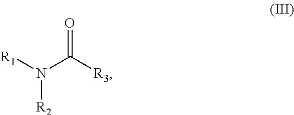

wherein R1, R2, and R3 are independently selected from H, unsubstituted or substituted alkyl, and unsubstituted or substituted aryl. In some embodiments, the formulation comprises an amide selected from the group consisting of N,N-dimethylpropionamide, N,N-dimethylacetamide, butyramide, and dimethylformamide. In some embodiments, the amide is N,N-dimethylpropionamide. In some embodiments, the amide is N,N-dimethylacetamide. In some embodiments, the amide is dimethylisobutyramide. In some embodiments, the amide is methylpropionamide. In some embodiments, the formulation comprises polyethylene glycol. In some embodiments, the polyethylene glycol is PEG 8000. In some embodiments, the polyethylene glycol is PEG 400. In some embodiments, the formulation is selected from formulations 14-21 of Table 3.

Described herein, in some embodiments, are admixtures comprising: (a) a formulation for substantially stable storage of one or more component of a PCR or RT-PCR reaction mixture at ambient temperatures, comprising: a hydroxyacid, and a borate composition; and (b) one or more component of a PCR or RT-PCR reaction mixture; wherein the one or more component of the PCR or RT-PCR reaction mixture remains stabilized after storage at ambient temperatures for a period of at least three months. In some embodiments, the hydroxyacid is a compound of formula (I):

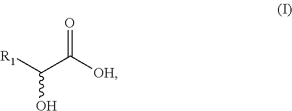

wherein R1 is selected from H, unsubstituted or substituted alkyl, or unsubstituted or substituted aryl. In some embodiments, the hydroxyacid is selected from the group consisting of lactic acid, malic acid, malonic acid, tartaric acid, citric acid, and combinations thereof. In some embodiments, the hydroxyacid is lactic acid. In some embodiments, the borate composition is selected from the group consisting of boric acid, borate, sodium tetraborate, borax, and a combination thereof. In some embodiments, the borate composition is selected from the group consisting of boric acid, sodium tetraborate, and a combination thereof. In some embodiments, the formulation comprises lactic acid and boric acid. In some embodiments, the formulation comprises lactic acid, boric acid, and sodium tetraborate. In some embodiments, the one or more component of a PCR or RT-PCR reaction mixture comprises a control nucleic acid. In some embodiments, the one or more component of a PCR or RT-PCR reaction mixture comprises an armored RNA. In some embodiments, the ARMORED RNA is a single strand of HIV RNA coated with the coat protein of MS2 bacteriophage, allowing the protection of the RNA strand. In some embodiments, the one or more component of a PCR or RT-PCR reaction mixture comprises an internal control template. In some embodiments, the polyethylene glycol is PEG 400. In some embodiments, the formulation is selected from formulations of Table 2.

Described herein, in some embodiments, are admixtures comprising: (a) a formulation for substantially stable storage of one or more component of a PCR or RT-PCR reaction mixture at ambient temperatures, comprising: a non-reducing sugar, and a buffer; and (b) one or more component of a PCR or RT-PCR reaction mixture; wherein the one or more component of the PCR or RT-PCR reaction mixture remains stabilized after storage at ambient temperatures for at least three months. In some embodiments, the non-reducing sugar is selected from the group consisting of sucrose and trehalose. In some embodiments, the non-reducing sugar is sucrose. In some embodiments, the buffer is selected from the group consisting of Tris-HCl, citric acid, tartaric acid, malic acid, sulfosalicylic acid, sulfoisophthalic acid, oxalic acid, borate, CAPS (3-(cyclohexylamino)-1-propanesulfonic acid), CAPSO (3-(cyclohexylamino)-2-hydroxy-1-propanesulfonic acid), EPPS (4-(2-hydroxyethyl)-1-piperazinepropanesulfonic acid), HEPES (4-(2-hydroxyethyl)piperazine-1-ethanesulfonic acid), MES (2-(N-morpholino)ethanesulfonic acid), MOPS (3-(N-morpholino)propanesulfonic acid), MOPSO (3-morpholino-2-hydroxypropanesulfonic acid), PIPES (1,4-piperazinediethanesulfonic acid), TAPS (N-[tris(hydroxymethyl)methyl]-3-aminopropanesulfonic acid), TAPSO (2-hydroxy-3-[tris(hydroxymethyl)methylamino]-1-propanesulfonic acid), TES (N-[tris(hydroxymethyl)methyl]-2-aminoethanesulfonic acid), bicine (N,N-bis(2-hydroxyethyl)glycine), tricine (N-[tris(hydroxymethyl)methyl]glycine), tris (tris(hydroxymethyl)aminomethane), and bis-tris (2-[bis(2-hydroxyethyl)amino]-2-(hydroxymethyl)-1,3-propanediol). In some embodiments, the formulation comprises sucrose and Tris-HCl. In some embodiments, the one or more component of a PCR or RT-PCR reaction mixture comprises a polymerase. In some embodiments, the one or more component of a PCR or RT-PCR reaction mixture comprises a Taq polymerase. In some embodiments, the one or more component of a PCR or RT-PCR reaction mixture comprises a PCR or RT-PCR master mix. In some embodiments, the PCR or RT-PCR master mix comprises enzymes, deoxynucleosidetriphosphates (dNTPs), buffer, salt, aptamer, primers, probes, and other additives. In some embodiments, the PCR or RT-PCR master mix comprises a polymerase, dNTPs, primers, and probes. In some embodiments, the PCR or RT-PCR master mix comprises a reverse transcriptase, a Taq polymerase, deoxynucleosidetriphosphates (dNTPs), primers, probes, buffers, salts, detergent, glycerol, DMSO, and one or more additional enzymes. In some embodiments, the Taq polymerase has reverse transcriptase activity. In some embodiments, the PCR or RT-PCR master mix comprises UNG. In some embodiments, the one or more component of a PCR or RT-PCR reaction mixture comprises an internal control template nucleic acid. In some embodiments, the polyethylene glycol is PEG 400. In some embodiments, the formulation is selected from formulations 12 or 13 of Table 3.

Described herein, in some embodiments, are admixtures comprising:(a) a formulation for substantially stable storage of one or more component of a PCR or RT-PCR reaction mixture at ambient temperatures, comprising: i) an aminosulfonic acid or ammonium sulfate, and ii) at least one of a non-reducing sugar, an amide, or polyethylene glycol; and (b) one or more component of a PCR or RT-PCR reaction mixture; wherein the one or more component of the PCR or RT-PCR reaction mixture remains stabilized after storage at ambient temperatures for a period of at least three months. In some embodiments, the formulation comprises an aminosulfonic acid of formula (II):

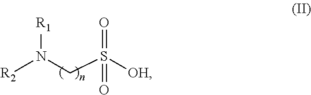

wherein R1 and R2 are independently selected from H, unsubstituted or substituted alkyl, and unsubstituted or substituted aryl. In some embodiments, the aminosulfonic acid is taurine. In some embodiments, the formulation comprises ammonium sulfate. In some embodiments, the formulation comprises a non-reducing sugar selected from the group consisting of sucrose and trehalose. In some embodiments, the non-reducing sugar is sucrose. In some embodiments, the formulation comprises an amide of formula (III):

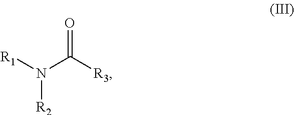

wherein R1, R2, and R3 are independently selected from H, unsubstituted or substituted alkyl, and unsubstituted or substituted aryl. In some embodiments, the formulation comprises an amide selected from the group consisting of N,N-dimethylpropionamide, N,N-dimethylacetamide, butyramide, and dimethylformamide. In some embodiments, the amide is N,N-dimethylpropionamide. In some embodiments, the amide is N,N-dimethylacetamide. In some embodiments, the formulation comprises dimethylisobutyramide. In some embodiments, the formulation comprises methylpropionamide. In some embodiments, the formulation comprises polyethylene glycol. In some embodiments, the polyethylene glycol is PEG 8000 or PEG 400. In some embodiments, the one or more component of a PCR or RT-PCR reaction mixture comprises a polymerase. In some embodiments, the one or more component of a PCR or RT-PCR reaction mixture comprises a Taq polymerase. In some embodiments, the one or more component of a PCR or RT-PCR reaction mixture comprises a PCR or RT-PCR master mix. In some embodiments, the PCR or RT-PCR master mix comprises enzymes, deoxynucleosidetriphosphates (dNTPs), buffer, salt, aptamer, primers, probes, and other additives. In some embodiments, the PCR or RT-PCR master mix comprises a polymerase, dNTPs, primers, and probes. In some embodiments, the PCR or RT-PCR master mix comprises a reverse transcriptase, a Taq polymerase, deoxynucleosidetriphosphates (dNTPs), primers, probes, buffers, salts, detergent, glycerol, DMSO, and one or more additional enzymes. In some embodiments, the Taq polymerase has reverse transcriptase activity. In some embodiments, the PCR or RT-PCR master mix comprises UNG. In some embodiments, the one or more component of a PCR or RT-PCR reaction mixture comprises an internal control template nucleic acid. In some embodiments, the polyethylene glycol is PEG 400. In some embodiments, the formulation is selected from formulations 14-21 of Table 3.

Described herein, in some embodiments, are admixtures comprising: (a) a formulation for substantially stable storage of one or more component of a PCR or RT-PCR reaction mixture at ambient temperatures, comprising: lactic acid; and (b) one or more component of a PCR or RT-PCR reaction mixture; wherein the one or more component of the PCR or RT-PCR reaction mixture remains stabilized after storage at ambient temperatures for a period of at least three months. In some embodiments, the lactic acid is present in the formulation at a concentration of 5% to 20%. In some embodiments, the lactic acid is present in the formulation at a concentration of 7% to 15%. In some embodiments, the formulation further comprises boric acid, sodium tetraborate, or a combination thereof. In some embodiments, the one or more component of a PCR or RT-PCR reaction mixture comprises a control nucleic acid. In some embodiments, the one or more component of a PCR or RT-PCR reaction mixture comprises an armored RNA.

Described herein, in some embodiments, are compositions comprising a substantially, stably stored one or more component of a PCR or RT-PCR reaction mixture admixed with a formulation for substantially stable storage of one or more component of a PCR or RT-PCR reaction mixture provided herein.

Described herein, in some embodiments, are articles of manufacture, comprising a formulation for substantially stable storage of one or more component of a PCR or RT-PCR reaction mixture provided herein.

Described herein, in some embodiments, are kits comprising an article of manufacture provided herein and a package insert.

Described herein, in some embodiments, are methods for substantially stable storage of one or more component of a PCR or RT-PCR reaction mixture at ambient temperatures, comprising: admixing a sample of one or more component of a PCR or RT-PCR reaction mixture with a formulation for substantially stable storage of one or more component of a PCR or RT-PCR reaction mixture provided herein, wherein the one or more component of a PCR or RT-PCR reaction mixture remains stabilized after storage at ambient temperatures for a period of at least three months. In some embodiments, at least 80% of the one or more component of a PCR or RT-PCR reaction mixture remains stabilized at ambient temperatures for a period of at least three months. In some embodiments, the one or more component of a PCR or RT-PCR reaction mixture is a RT-PCR master mix, a RT-PCR internal control template nucleic acid, or a combination thereof.

Described herein, in some embodiments, are methods for substantially stable storage of one or more component of a PCR or RT-PCR reaction mixture at ambient temperatures, comprising: admixing one or more component of a PCR or RT-PCR reaction mixture with a formulation selected from the group consisting of: (a) a formulation comprising a hydroxyacid, and a borate composition; (b) a formulation comprising a non-reducing sugar and a buffer; and (c) a formulation comprising i) an aminosulfonic acid or ammonium sulfate, and ii) at least one of a non-reducing sugar, an amide, or polyethylene glycol;, wherein the one or more component of a PCR or RT-PCR reaction mixture remains stabilized after storage at ambient temperatures for a period of at least three months. In some embodiments, at least 80% of the one or more component of a PCR or RT-PCR reaction mixture remains stabilized at ambient temperatures for a period of at least three months. In some embodiments, the one or more component of a PCR or RT-PCR reaction mixture is a RT-PCR master mix, a RT-PCR internal control template nucleic acid, or a combination thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-D illustrate stabilization of ARMORED RNA (aRNA; Roche). aRNA was diluted and mixed with either water (4C Ctrl and no formulation control (NFC)) or with formulation 01 as described in the experimental protocol (Example 1). The “4C Ctrl” sample was stored at 4° C. Sample “NFC,” no formulation control, and sample containing formulation 01 were both stored at either 45° C. (FIG. 1A and FIG. 1C) or 37° C. (FIG. 1B and FIG. 1D) for the indicated times. At the noted times, 10 μl of sample was removed and processed as described in the experimental protocol (Example 1). Processed RNA templates were used in the HIV real time RT-PCR assay. FIG. 1A and FIG. 1B display the average cycle threshold (Ct) for each sample. FIG. 1C and FIG. 1D display the average relative fluorescent units (RFU) for the same samples collected from the real time RT-PCR. Data show that the samples containing formulation 01 have lower Ct values and improved fluorescence compared to the NFC, indicating an increased amount of RNA yield from those samples.

FIGS. 2A-B illustrate stabilization of ARMORED RNA (aRNA; Roche). aRNA was diluted and mixed with either water (4C Ctrl and NFC) or with formulation 02 as described in the experimental protocol (Example 1). The “4C Ctrl” sample was stored at 4° C. Sample “NFC,” no formulation control, and sample containing formulation 02 were both stored at either 45° C. (FIG. 2A) or 37° C. (FIG. 2B) for the indicated times. At the noted times, 10 μl of sample was removed and processed as described in the experimental protocol (Example 1). Processed RNA templates were used in the HIV real time RT-PCR assay. FIG. 2A and FIG. 2B display the average cycle threshold (Ct) for each sample collected from the real time RT-PCR. Data show that the samples containing formulation 02 have lower Ct values compared to the NFC, indicating an increased amount of RNA yield from those samples.

FIGS. 3A-B illustrate stabilization of ARMORED RNA (aRNA; Roche). aRNA was diluted and mixed with either water (4C Ctrl and NFC) or with the listed formulation as described in the experimental protocol (Example 1). The “4C Ctrl” sample was stored at 4° C. Sample “NFC,” no formulation control, and samples with formulation were all stored at 45° C. for the indicated times. At the noted times, 10 μl of sample was removed and processed as described in the experimental protocol (Example 1). Processed RNA templates were used in the HIV real time RT-PCR assay. Panels display the average cycle threshold (Ct) for each sample collected from the real time RT-PCR. Data show that the samples containing formulations 01, 02, and 04 have lower Ct values compared to the NFC, indicating an increased amount of RNA yield from those samples. All other formulations tested in these experiments show Ct values at or above those for the NFC, indicating no improved RNA yield or stability. FIG. 3A illustrates storage for 19 days at 45° C.; FIG. 3B illustrates storage for 26 days at 45° C.

FIGS. 4A-C illustrate stabilization of the complete RT-PCR mastermix (Roche). A 1.5x concentrated RT-PCR mastermix was mixed with either water (4C Ctrl or NFC) or with the indicated formulation as described in the experimental protocol (Example 2). The “4C Ctrl” sample was stored at 4° C. Sample “NFC,” no formulation control, and samples with formulations were stored at 50° C. for 14 days. At that time, samples were removed and combined with remaining necessary components to run the HIV real time RT-PCR assay, as described in the experimental protocol (Example 2). The panels display the average cycle threshold (Ct) for each sample collected from the real time RT-PCR. FIG. 4A displays results obtained for formulations 12 and 13. FIG. 4B displays results obtained for formulations 14-17. FIG. 4C displays results obtained for formulations 18-21. Data show that the samples containing the indicated formulations have lower Ct values for both RT-PCR targets (HIV and QS RNA) compared to the NFC.

### DETAILED DESCRIPTION OF THE INVENTION

The present invention relates to formulations, compositions, articles of manufacture, kits, and methods for substantially stable storage of one or more component of a PCR or RT-PCR reaction mixture at ambient temperatures.

Provided herein are formulations, methods, and compositions for the stabilization of one or more reagents for a polymerase chain reaction (PCR) assay. PCR reagents may be stored individually or in combination for use in methods such as nucleic acid amplification and sequencing. The formulations, methods, and compositions provide for long term storage of PCR reagents at room temperature. In addition, in some embodiments, the formulations, methods, and compositions provide for stable storage of PCR reagents in liquid form.

In some embodiments, the formulations disclosed herein provide for long term storage of one or more component of a PCR or RT-PCR reaction mixture. In some embodiments, the one or more component of a PCR or RT-PCR reaction mixture comprises a polymerase. In some embodiments, the one or more component of a PCR or RT-PCR reaction mixture comprises a Taq polymerase. In some embodiments, the one or more component of a PCR or RT-PCR reaction mixture comprises a PCR or RT-PCR master mix. In some embodiments, the PCR or RT-PCR master mix comprises enzymes, deoxynucleoside triphosphates (dNTPs), buffer, salt, aptamer, primers, probes, and other additives. In some embodiments, the PCR or RT-PCR master mix comprises a polymerase, dNTPs, primers, and probes. In some embodiments, the one or more component of a PCR or RT-PCR reaction mixture comprises an internal control template nucleic acid.

An internal control template nucleic acid may be RNA or DNA. In some embodiments, an internal control template serves as a quantification standard for PCR and RT-PCR assays. In some embodiments, the internal control template is an ARMORED RNA (Pasloske et al., J. Clin. Microbiol. 1998; 36(12):3590-3594; U.S. Pat. No. 5,677,124; Roche). In some embodiments, the ARMORED RNA is principally made of a single strand of HIV RNA coated with the coat protein of MS2 bacteriophage, allowing the protection of the RNA strand.

In some embodiments, the PCR or RT-PCR assay is an assay for the detection and quantification of infectious agents. In some embodiments, the infectious agent is a virus. In some embodiments, the infectious agent is HIV.

### Definitions

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

“About” as used herein when referring to a measurable value such as an amount, a temporal duration, and the like, is meant to encompass variations of ±20% or ±10%, or ±5%, or even ±1% from the specified value, as such variations are appropriate for the disclosed compositions or to perform the disclosed methods.

Unless defined otherwise, all technical and scientific terms used herein have the same meaning as is commonly understood by one of skill in the art to which this invention belongs.

The term “ambient temperature” as used herein refers to common indoor room temperatures. In some embodiments, ambient temperature is 15 to 32° C. In some embodiments, ambient temperature is 20 to 27° C.

An “alkyl” group refers to an aliphatic hydrocarbon group. The alkyl moiety includes a “saturated alkyl” group, which means that it does not contain any alkene or alkyne moieties. The alkyl moiety also includes an “unsaturated alkyl” moiety, which means that it contains at least one alkene or alkyne moiety. An “alkene” moiety refers to a group that has at least one carbon-carbon double bond, and an “alkyne” moiety refers to a group that has at least one carbon-carbon triple bond. The alkyl moiety, whether saturated or unsaturated, includes branched, straight chain, or cyclic moieties. Depending on the structure, an alkyl group includes a monoradical or a diradical (i.e., an alkylene group), and if a “lower alkyl” having 1 to 6 carbon atoms. As used herein, C1-Cx includes C1-C2, C1-C3 . . . C1-Cx. The “alkyl” moiety optionally has 1 to 10 carbon atoms (whenever it appears herein, a numerical range such as “1 to 10” refers to each integer in the given range; e.g., “1 to 10 carbon atoms” means that the alkyl group is selected from a moiety having 1 carbon atom, 2 carbon atoms, 3 carbon atoms, etc., up to and including 10 carbon atoms, although the present definition also covers the occurrence of the term “alkyl” where no numerical range is designated). The alkyl group of the compounds described herein may be designated as “C1-C4 alkyl” or similar designations. By way of example only, “C1-C4 alkyl” indicates that there are one to four carbon atoms in the alkyl chain, i.e., the alkyl chain is selected from among methyl, ethyl, propyl, iso-propyl, n-butyl, iso-butyl, sec-butyl, and t-butyl. Thus C1-C4 alkyl includes C1-C2 alkyl and C1-C3 alkyl. Alkyl groups are optionally substituted or unsubstituted. Typical alkyl groups include, but are in no way limited to, methyl, ethyl, propyl, isopropyl, butyl, isobutyl, tertiary butyl, pentyl, hexyl, ethenyl, propenyl, butenyl, cyclopropyl, cyclobutyl, cyclopentyl, cyclohexyl, and the like.

An “amide” is a chemical moiety with the formula —C(O)NHR or —NHC(O)R, where R is selected from among alkyl, cycloalkyl, aryl, heteroaryl (bonded through a ring carbon) and heteroalicyclic (bonded through a ring carbon). The procedures and specific groups to make such amides are known to those of skill in the art and can readily be found in reference sources such as Greene and Wuts, Protective Groups in Organic Synthesis, 3^{rd} Ed., John Wiley & Sons, New York, N.Y., 1999, which is incorporated herein by reference in its entirety.

The term “aryl” used alone or as part of a larger moiety as in “arylalkyl”, “arylalkoxy”, or “aryloxyalkyl”, refers to a monocyclic, bicyclic or tricyclic, carbon ring system, that includes fused rings, wherein at least one ring in the system is aromatic. The term “aryl” may be used interchangeably with the term “aryl ring”. In one embodiment, aryl includes groups having 6-12 carbon atoms. In another embodiment, aryl includes groups having 6-10 carbon atoms. Examples of aryl groups include phenyl, naphthyl, anthracyl, phenanthrenyl, naphthacenyl, 1,2,3,4-tetrahydronaphthalenyl, 1H-indenyl, 2,3-dihydro-1H-indenyl, and the like. A particular aryl is phenyl. In another embodiment aryl includes indanyl, naphthyl, and tetrahydronaphthyl, and the like, where the radical or point of attachment is on an aromatic ring.

The term “optionally substituted” or “substituted” means that the referenced group may be substituted with one or more additional group(s) individually and independently selected from alkyl, cycloalkyl, aryl, heteroaryl, heteroalicyclic, hydroxy, alkoxy, aryloxy, alkylthio, arylthio, alkylsulfoxide, arylsulfoxide, alkylsulfone, arylsulfone, cyano, halo, acyl, nitro, haloalkyl, fluoroalkyl, amino, including mono- and di-substituted amino groups, and the protected derivatives thereof. By way of example, optional substituents may be LsRs, wherein each Ls is independently selected from a bond, —O—, —C(═O)—, —S—, —S(═O)—, —S(═O)₂—, —NH—, —NHC(O)—, —C(O)NH—, S(═O)₂NH—, —NHS(═O)₂, —OC(O)NH—, —NHC(O)O—, -(substituted or unsubstituted C1-C6 alkyl), or -(substituted or unsubstituted C2-C6 alkenyl); and each Rs is independently selected from H, (substituted or unsubstituted C1-C4 alkyl), (substituted or unsubstituted C3-C6 cycloalkyl), heteroaryl, or heteroalkyl.

Definition of standard chemistry terms may be found in reference works, including Carey and Sundberg “ADVANCED ORGANIC CHEMISTRY 4TH ED.” Vols. A (2000) and B (2001), Plenum Press, New York. Unless otherwise indicated, conventional methods of mass spectroscopy, NMR, HPLC, protein chemistry, biochemistry, recombinant DNA techniques and pharmacology, within the skill of the art are employed. Unless specific definitions are provided, the nomenclature employed in connection with, and the laboratory procedures and techniques of, analytical chemistry, synthetic organic chemistry, and medicinal and pharmaceutical chemistry described herein are those known in the art. Standard techniques can be used for chemical syntheses, chemical analyses, pharmaceutical preparation, formulation, and delivery, and treatment of patients. Standard techniques can be used for recombinant DNA, oligonucleotide synthesis, and tissue culture and transformation (e.g., electroporation, lipofection). Reactions and purification techniques can be performed e.g., using kits of manufacturer's specifications or as commonly accomplished in the art or as described herein. The foregoing techniques and procedures can be generally performed of conventional methods well known in the art and as described in various general and more specific references that are cited and discussed throughout the present specification.

As used herein, the substituent “R” appearing by itself and without a number designation refers to a substituent selected from among from alkyl, cycloalkyl, aryl, heteroaryl (bonded through a ring carbon) and non-aromatic heterocycle (bonded through a ring carbon).

As used herein, the terms “stabilization,” “stabilizing,” and “stabilized,” when used in reference to one or more component of a PCR or RT-PCR reaction mixture refer to the ability of a material to maintain, enhance, or otherwise inhibit the decline or loss of the activity of the one or more component of a PCR or RT-PCR reaction mixture, often as measured over time (i.e., in the presence of a stabilizer, the one or more component of a PCR or RT-PCR reaction mixture retains its activity for a longer time period than in the absence of the stabilizer). Activity of the one or more component of a PCR or RT-PCR reaction mixture is measured by performing a PCR or RT-PCR assay according to methods known in the art. “Stabilization of one or more component of a PCR or RT-PCR reaction mixture” also refers to the ability of a material to maintain activity under suboptimal conditions of temperature or pH. As another example, “stabilizing one or more component of a PCR or RT-PCR reaction mixture” refers to the ability of a material to enhance activity under suboptimal conditions, as compared to activity in the absence of a “stabilizing” compound or material.

The term “polymerase” refers to an enzyme that synthesizes nucleic acid strands (e.g., RNA or DNA) from ribonucleoside triphosphates or deoxynucleoside triphosphates.

As used herein, “nucleic acid” refers to both, a deoxyribonucleic acid (DNA) and a ribonucleic acid (RNA), as well as modified and/or functionalized versions thereof. Similarly, the term “nucleotide” as used herein includes both individual units of ribonucleic acid and deoxyribonucleic acid as well as nucleoside and nucleotide analogs, and modified nucleotides such as labeled nucleotides. In addition, “nucleotide” includes non-naturally occurring analogue structures, such as those in which the sugar, phosphate, and/or base units are absent or replaced by other chemical structures. Thus, the term “nucleotide” encompasses individual peptide nucleic acid (PNA) (Nielsen et al., Bioconjug. Chem. 1994; 5(1):3-7) and locked nucleic acid (LNA) (Braasch and Corey, Chem. Biol. 2001; 8(1):1-7) units as well as other like units.

As used herein, the term “QS” refers to an RNA that is used as a quantification standard in an RT-PCR reaction.

As used herein, the terms “aRNA” and “QS aRNA” refer to an ARMORED RNA (Roche) that is used as a quantification standard in an RT-PCR reaction.

As described herein, the term “polypeptide” refers to any polymeric chain of amino acids. The terms “peptide” and “protein” are used interchangeably with the term polypeptide and also refer to a polymeric chain of amino acids. The term “polypeptide” encompasses native or artificial proteins, protein fragments and polypeptide analogs of a protein sequence. A polypeptide may be monomeric or polymeric. The term “polypeptide” encompasses fragments and variants (including fragments of variants) thereof, unless otherwise contradicted by context.

### Formulation Reagents

### pH Buffers

According to certain embodiments, the herein described formulations and compositions for substantially stable storage of one or more component of a PCR or RT-PCR reaction mixture include one or more pH buffers. In some embodiments, the pH buffer is any of a large number of compounds known in the art for their ability to resist changes in the pH of a solution, such as an aqueous solution, in which the pH buffer is present. Selection of one or more particular pH buffers for inclusion in a stable storage composition may be done based on the present disclosure and according to routine practices in the art, and may be influenced by a variety of factors including the pH that is desirably to be maintained, the nature of the sample to be stabilized, the solvent conditions to be employed, the other components of the formulation to be used, and other criteria. For example, typically a pH buffer is employed at a pH that is within about 0.1, 0.2, 0.3, 0.4, 0.5, 0.6, 0.7, 0.8, 0.9 or 1.0 pH unit of a proton dissociation constant (pKa) that is a characteristic of the buffer.

Non-limiting examples of pH buffers include Tris-HCl, citric acid, tartaric acid, malic acid, sulfosalicylic acid, sulfoisophthalic acid, oxalic acid, borate, CAPS (3-(cyclohexylamino)-1-propanesulfonic acid), CAPSO (3-(cyclohexylamino)-2-hydroxy-1-propanesulfonic acid), EPPS (4-(2-hydroxyethyl)-1-piperazinepropanesulfonic acid), HEPES (4-(2-hydroxyethyl)piperazine-1-ethanesulfonic acid), MES (2-(N-morpholino)ethanesulfonic acid), MOPS (3-(N-morpholino)propanesulfonic acid), MOPSO (3-morpholino-2-hydroxypropanesulfonic acid), PIPES (1,4-piperazinediethanesulfonic acid), TAPS (N-[tris(hydroxymethyl)methyl]-3-aminopropanesulfonic acid), TAPSO (2-hydroxy-3-[tris(hydroxymethyl)methylamino]-1-propanesulfonic acid), TES (N-[tris(hydroxymethyl)methyl]-2-aminoethanesulfonic acid), bicine (N,N-bis(2-hydroxyethyl)glycine), tricine (N-[tris(hydroxymethyl)methyl]glycine), tris (tris(hydroxymethyl)aminomethane), and bis-tris (2-[bis(2-hydroxyethyl)amino]-2-(hydroxymethyl)-1,3-propanediol). Certain embodiments contemplated herein, including those set forth in Table 1, feature a formulation having a pH of about 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9 or 9.0. In some embodiments, the formulation has a pH of about 7 to 9.

### Organic Acids

In some embodiments, an organic acid is included in the presently described formulations and compositions for substantially stable storage one or more component of a PCR or RT-PCR reaction mixture. Organic acids include, for example, hydroxyacids, carboxylic acids, and aminosulfonic acids.

In some embodiments, the organic acid is a hydroxyacid. In some embodiments, the hydroxyacid is a compound of formula (I):

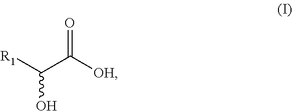

wherein R1 is selected from H, unsubstituted or substituted alkyl, or unsubstituted or substituted aryl. In some embodiments, the hydroxyacid is selected from the group consisting of lactic acid, malic acid, malonic acid, tartaric acid, citric acid, and a combination thereof. In some embodiments, the hydroxyacid is lactic acid. In some embodiments, the hydroxyacid is present in the formulation at a concentration of 5% to 20%. In some embodiments, the hydroxyacid is present in the formulation at a concentration of 7% to 15%.

In some embodiments, the organic acid is an aminosulfonic acid. In some embodiments, the aminosulfonic acid is a compound of formula (II):

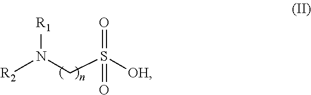

wherein R1 and R2 are independently selected from H, unsubstituted or substituted alkyl, and unsubstituted or substituted aryl. In some embodiments, the aminosulfonic acid is taurine. Taurine is an amino acid that is not incorporated into proteins. In some embodiments, the organic acid is lactic acid, taurine, or both.

In some embodiments, the organic acid is a carboxylic acid. In some embodiments, the carboxylic acid is hydroxyectoine.

In some embodiments, the organic acid is an amino acid. Amino acids include the natural amino acids alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, and their derivatives. An example of an amino acid derivative is betaine. In some embodiments, the organic acid is betaine.

### Amides

The herein described formulations for substantial stable storage of one or more component of a PCR or RT-PCR reaction mixture at ambient temperatures may, in certain embodiments, contain an amide. In some embodiments, the amide is a compound of formula (III):

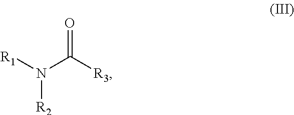

wherein R1, R2, and R3 are independently selected from H, unsubstituted or substituted alkyl, and unsubstituted or substituted aryl. In some embodiments, the amide is selected from the group consisting of N,N-dimethylpropionamide, N,N-dimethylacetamide, butyramide, and dimethylformamide. In some embodiments, the amide is N,N-dimethylpropionamide. In some embodiments, the amide is N,N-dimethylacetamide.

### Polyethers

The herein described formulations for substantial stable storage of one or more component of a PCR or RT-PCR reaction mixture at ambient temperatures may, in certain embodiments, contain a polyether. Polyethers are polymers that contain more than one ether functional group. Polyethers include, for example, polyethylene glycol (PEG), polyethylene oxide (PEO), polyoxyethylene (POE), polypropylene glycol (PPG), polytetramethylene glycol (PTMG), polytetramethylene ether glycol (PTMEG), and paraformaldehyde. Aromatic polyethers include, for example, polyphenyl ether (PPE) and poly(p-phenylene oxide) (PPO). In some embodiments, the polyether is polyethylene glycol (PEG), polyethylene oxide (PEO), or polyoxyethylene (POE). In some embodiments, the polyether is polyethylene glycol (PEG). The molecular weight of polyethylene glycol (PEG) may range from 300 g/mol to 10,000,000 g/mol. In some embodiments, the polyether is PEG 8000. In some embodiments, the polyether is PEG 400.

### Chelating Agents

Chelating agents or chelators are, according to certain embodiments, included in the presently described formulations and compositions for substantially stable storage of one or more component of a PCR or RT-PCR reaction mixture. Such chelating agents are known to those familiar with the art for their ability to complex with and hinder the reactivity of metal cations. Exemplary chelating agents include diethylenetriaminepentaacetic acid (DTPA), ethylenediaminetetraacetic acid (EDTA), ethylene glycol tetraacetic acid (EGTA), trans-1,2-diaminocyclohexane-N,N,N′,N′-tetraacetic acid (CDTA), 1,2-bis(2-aminophenoxy)ethane-N,N,N′,N′-tetraacetic acid (BAPTA), 1,4,7,10-tetraazacyclododecane-1,4,7,10-tetraacetic acid (DOTA), N-(2-hydroxyethyl)ethylenediamine-N,N′,N′-triacetic acid, sodium gluconate, and nitrilotriacetic acid (NTA). In some embodiments, the chelating agent is EDTA. In some embodiments, the chelating agent is present at a concentration of about 0.01-0.1 mM, about 0.1-1.0 mM, about 1.0-50 mM, or about 10-40 mM, or about 25 mM.

### Non-Reducing Sugars

In some embodiments, the formulations and compositions for substantially stable storage of one or more component of a PCR or RT-PCR reaction mixture at ambient temperatures include at least one non-reducing sugar. As used herein, “non-reducing sugars” refers to carbohydrate molecules that lack a functional aldehyde group. Exemplary non-reducing sugars include sucrose and trehalose. In some embodiments, the non-reducing sugar is sucrose. In some embodiments, the non-reducing sugar is trehalose. In some embodiments, the non-reducing sugar is present at a concentration of about 1.0-50 mM. In some embodiments, the non-reducing sugar is present at a concentration of about 10.0-30 mM. In some embodiments, the non-reducing sugar is present at a concentration of about 25 mM. In some embodiments, the non-reducing sugar is present at a concentration of about 25-100 mM, about 100-500 mM, about 200-700 mM, about 300-800 mM, about 400-900 mM, or about 500-1000 mM. In some embodiments, the non-reducing sugar is present at a concentration of about 400-600 mM. In some embodiments, the non-reducing sugar is present at a concentration of about 1% to about 50% (weight/volume).

### Detergents or Surfactants

In certain embodiments, the formulations for substantially stable storage of one or more component of a PCR or RT-PCR reaction mixture at ambient temperatures contain a detergent or surfactant. In certain embodiments, the detergent or surfactant is selected from TRITON X-100 4-(1,1,3,3-tetramethylbutyl)phenyl-polyethylene glycol, NONIDET P-40 polyethylene glycol mono(octylphenyl) ether, any of the BRIJ family of detergents, any of the TWEEN family of surfactants, sodium dodecyl sulfate, sodium lauryl sulfate, deoxycholate, octyl-glucopyranoside, betaines, or the like. Certain contemplated embodiments, including those that may relate to specific types of biological samples, expressly exclude the presence of a denaturing detergent when a chelating agent is also present, and in particular of a detergent of a type and at a concentration sufficient to denature a protein, polypeptide or nucleic acid molecule, while certain other contemplated embodiments are not so limited, where it is to be noted that many detergents and surfactants are non-denaturing for many if not all types of biological samples.

In other embodiments, the detergent or surfactant may be a TOMAMINE detergent. Examples of TOMAMINE detergents include bis-(2-hydroxyethyl) isodecycloxypropylamine, poly (5) oxyethylene isodecycloxypropylamine, bis-(2-hydroxyethyl) isotridecycloxypropylamine, poly (5) oxyethylene isotridecycloxypropyl amine, bis-(2-hydroxyethyl) linear alkyloxypropylamine, bis (2-hydroxyethyl) soya amine, poly (15) oxyethylene soya amine, bis (2-hydroxyethyl) octadecyclamine, poly (5) oxyethylene octadecylamine, poly (8) oxyethylene octadecylamine, poly (10) oxyethylene octadecylamine, poly (15) oxyethylene octadecylamine, bis(2-hydroxyethyl) octadecycloxypropylamine, bis-(2-hydroxyethyl) tallow amine, poly (5) oxyethylene tallow amine, poly (15) oxyethylene tallow amine, poly (3) oxyethylene 1,3 diaminopropane, bis(2-hyrdoxyethyl) coco amine, bis-(2-hydroxyethyl) isodecycloxypropylamine, poly (5) oxy-ethylene isodecycloxypropylamine, bis-(2-hydroxyethyl) isotridecycloxypropylamine, poly (5) oxyethylene isotridecyloxypropylamine, bis-(2-hydroxyethyl) linear alkyloxypropylamine, bis(2-hydroxyethyl) soya amine, poly (15) oxyethylene soya amine, bis(2-hydroxyethyl) octadecylamine, poly (5) oxyethylene octadecylamine, poly (8) oxyethylene octadecylamine, poly (10) oxyethylene octadecylamine, poly (15) oxyethylene octadecylamine, bis(2-hydroxyethyl) octadecycloxypropylamine, bis-(2-hydroxyethyl) tallow amine, poly (5) oxyethylene tallow amine, poly (15) oxyethylene tallow amine, poly (3) oxyethylene 1,3 diaminopropane, and bis(2-hydroxyethyl) coco amine.

### Borate Compositions

In some embodiments, the borate composition comprises a compound selected from the group consisting of boric acid, borate, sodium tetraborate, borax, and a combination thereof. In some embodiments, the borate composition comprises a compound selected from the group consisting of boric acid, sodium tetraborate, and a combination thereof. In some embodiments, the borate composition comprises boric acid. In some embodiments, the borate composition comprises boric acid and sodium tetraborate. In some embodiments, the borate composition is present at a concentration of 2 mM to 300 mM. In some embodiments, the borate composition is present at a concentration of 5 mM to 50 mM.

### Exemplary Formulations for Stabilization of One or More Component of a PCR or RT-PCR Reaction Mixture at Ambient Temperatures

Described herein, in some embodiments, are formulations for substantially stable storage of one or more component of a PCR or RT-PCR reaction mixture at ambient temperatures, comprising: (i) a hydroxyacid; and (ii) a borate composition, wherein the one or more component of a PCR or RT-PCR reaction mixture remains stabilized after storage at ambient temperatures for a period of at least three months. In some embodiments, the hydroxyacid is selected from the group consisting of a compound of formula (I):

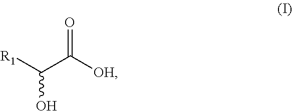

wherein R1 is selected from H, unsubstituted or substituted alkyl, and unsubstituted or substituted aryl. In some embodiments, the hydroxyacid is selected from the group consisting of lactic acid, malic acid, malonic acid, tartaric acid, citric acid, and a combination thereof. In some embodiments, the hydroxyacid is lactic acid. In some embodiments, the borate composition is selected from the group consisting of boric acid, borate, sodium tetraborate, borax, and a combination thereof. In some embodiments, the borate composition is selected from the group consisting of boric acid, sodium tetraborate, and a combination thereof. In some embodiments, the formulation comprises lactic acid and boric acid. In some embodiments, the formulation comprises lactic acid, boric acid, and sodium tetraborate.

Described herein, in some embodiments, are formulations for substantially stable storage of one or more component of a PCR or RT-PCR reaction mixture at ambient temperatures, comprising: (i) a non-reducing sugar; and (ii) a buffer, wherein the one or more component of a PCR or RT-PCR reaction mixture remains stabilized after storage at ambient temperatures for at least three months. In some embodiments, the non-reducing sugar is selected from the group consisting of sucrose and trehalose. In some embodiments, the non-reducing sugar is sucrose. In some embodiments, the buffer is selected from the group consisting of Tris-HCl, citric acid, tartaric acid, malic acid, sulfosalicylic acid, sulfoisophthalic acid, oxalic acid, borate, CAPS (3-(cyclohexylamino)-1-propanesulfonic acid), CAPSO (3-(cyclohexylamino)-2-hydroxy-1-propanesulfonic acid), EPPS (4-(2-hydroxyethyl)-1-piperazinepropanesulfonic acid), HEPES (4-(2-hydroxyethyl)piperazine-1-ethanesulfonic acid), MES (2-(N-morpholino)ethanesulfonic acid), MOPS (3-(N-morpholino)propanesulfonic acid), MOPSO (3-morpholino-2-hydroxypropanesulfonic acid), PIPES (1,4-piperazinediethanesulfonic acid), TAPS (N-[tris(hydroxymethyl)methyl]-3-aminopropanesulfonic acid), TAPSO (2-hydroxy-3-[tris(hydroxymethyl)methylamino]-1-propanesulfonic acid), TES (N-[tris(hydroxymethyl)methyl]-2-aminoethanesulfonic acid), bicine (N,N-bis(2-hydroxyethyl)glycine), tricine (N-[tris(hydroxymethyl)methyl]glycine), tris (tris(hydroxymethyl)aminomethane), and bis-tris (2-[bis(2-hydroxyethyl)amino]-2-(hydroxymethyl)-1,3-propanediol). In some embodiments, the formulation comprises Tris-HCl. In some embodiments, the formulation comprises sucrose and Tris-HCl.

Described herein, in some embodiments, are formulations for substantially stable storage of one or more component of a PCR or RT-PCR reaction mixture at ambient temperatures, comprising: (i) an aminosulfonic acid or ammonium sulfate; and (ii) at least one of a non-reducing sugar, an amide, or polyethylene glycol, wherein the one or more component of a PCR or RT-PCR reaction mixture remains stabilized after storage at ambient temperatures for a period of at least three months. In some embodiments, the formulation comprises an aminosulfonic acid selected from the group consisting of a compound of formula (II):

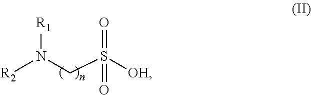

wherein R1 and R2 are independently selected from H, unsubstituted or substituted alkyl, and unsubstituted or substituted aryl. In some embodiments, the aminosulfonic acid is taurine. In some embodiments, n may be a number from one to six. In some embodiments, n may be any other number. In some embodiments, the formulation comprises ammonium sulfate. In some embodiments, the formulation comprises a non-reducing sugar selected from the group consisting of sucrose and trehalose. In some embodiments, the non-reducing sugar is sucrose. In some embodiments, the formulation comprises an amide selected from the group consisting of a compound of formula (III):

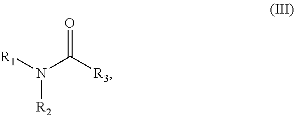

wherein R1, R2, and R3 are independently selected from H, unsubstituted or substituted alkyl, and unsubstituted or substituted aryl. In some embodiments, the formulation comprises an amide selected from the group consisting of N,N-dimethylpropionamide, N,N-dimethylacetamide, butyramide, and dimethylformamide. In some embodiments, the amide is N,N-dimethylpropionamide. In some embodiments, the amide is N,N-dimethylacetamide. In some embodiments, the amide is dimethylisobutyramide. In some embodiments, the amide is methylpropionamide. In some embodiments, the formulation comprises polyethylene glycol. In some embodiments, the polyethylene glycol is PEG 8000. In some embodiments, the polyethylene glycol is PEG 400.

Described herein, in some embodiments, are formulations for substantially stable storage of one or more component of a PCR or RT-PCR reaction mixture at ambient temperatures, comprising: (i) an aminosulfonic acid; and (ii) an amino acid, wherein the one or more component of a PCR or RT-PCR reaction mixture remains stabilized after storage at ambient temperatures for a period of at least three months. In some embodiments, the formulation comprises an aminosulfonic acid selected from the group consisting of a compound of formula (II):

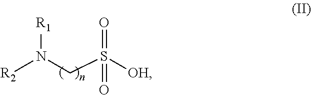

wherein R1 and R2 are independently selected from H, unsubstituted or substituted alkyl, and unsubstituted or substituted aryl. In some embodiments, the aminosulfonic acid is taurine. In some embodiments, the formulation comprises an amino acid selected from the group consisting of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, and betaine. In some embodiments, the amino acid is betaine. In some embodiments, the formulation comprises taurine and betaine.

Described herein, in some embodiments, are formulations for substantially stable storage of one or more component of a PCR or RT-PCR reaction mixture at ambient temperatures, comprising: (i) a carboxylic acid; and (ii) a buffer, wherein the one or more component of a PCR or RT-PCR reaction mixture remains stabilized after storage at ambient temperatures for a period of at least three months. In some embodiments, the carboxylic acid is hydroxyectoine. In some embodiments, the buffer is selected from the group consisting of Tris-HCl, citric acid, tartaric acid, malic acid, sulfosalicylic acid, sulfoisophthalic acid, oxalic acid, borate, CAPS (3-(cyclohexylamino)-1-propanesulfonic acid), CAPSO (3-(cyclohexylamino)-2-hydroxy-1-propanesulfonic acid), EPPS (4-(2-hydroxyethyl)-1-piperazinepropanesulfonic acid), HEPES (4-(2-hydroxyethyl)piperazine-1-ethanesulfonic acid), MES (2-(N-morpholino)ethanesulfonic acid), MOPS (3-(N-morpholino)propanesulfonic acid), MOPSO (3-morpholino-2-hydroxypropanesulfonic acid), PIPES (1,4-piperazinediethanesulfonic acid), TAPS (N-[tris(hydroxymethyl)methyl]-3-aminopropanesulfonic acid), TAPSO (2-hydroxy-3-[tris(hydroxymethyl)methylamino]-1-propanesulfonic acid), TES (N-[tris(hydroxymethyl)methyl]-2-aminoethanesulfonic acid), bicine (N,N-bis(2-hydroxyethyl)glycine), tricine (N-[tris(hydroxymethyl)methyl]glycine), tris (tris(hydroxymethyl)aminomethane), and bis-tris (2-[bis(2-hydroxyethyl)amino]-2-(hydroxymethyl)-1,3-propanediol). In some embodiments, the formulation comprises Tris-HCl. In some embodiments, the formulation comprises hydroxyectoine and Tris-HCl.

In some embodiments, the formulation for substantially stable storage of one or more component of a PCR or RT-PCR reaction comprises ions. In some embodiments, the ions are sodium ions or chlorine ions, or a combination thereof.

In some embodiments, the formulation for substantially stable storage of one or more component of a PCR or RT-PCR reaction comprises a chelating agent. In some embodiments, the chelating agent is EDTA.

In some embodiments, the formulation comprises a taurine variant. In some embodiments, the taurine variant is TES (N-[tris(hydroxymethyl)methyl]-2-aminoethanesulfonic acid).

In some embodiments, the formulation for substantially stable storage of one or more component of a PCR or RT-PCR reaction comprises a detergent. In some embodiments, the detergent is Tween 20 or Tween 80.

Exemplary formulations for substantially stable storage of one or more component of a PCR or RT-PCR reaction mixture at ambient temperatures are shown in Table 1.

TABLE 1
Exemplary Formulations for Stabilizing One or More
Component of a PCR or RT-PCR Reaction Mixture

| Name | Component 1 | Component 2 | Component 3 |
|---|---|---|---|
| 01 | 10% Lactic Acid | 50 mM Boric Acid | 10 mM Sodium |
| | | | Tetraborate, |
| | | | pH 5-8 |
| 02 | 7.5% Lactic Acid | 2% Boric Acid, pH 5-8 | |
| 04 | 10% Lactic Acid | | |
| 12 | 50% Sucrose | 50 mM Tris-HCl, pH 8 | |
| 13 | 50 mM Taurine | 900 mM Sucrose, pH 8-9 | |
| 14 | 50 mM Taurine | 5% Dimethylpropionamide, | |
| | | pH 8-9 | |
| 15 | 50 mM Taurine | 5% Dimethylacetamide, | |
| | | pH 8-9 | |
| 16 | 50 mM Taurine | 10% PEG 8000, pH 8-9 | |
| 17 | 50 mM Taurine | 10% PEG 400, pH 8-9 | |

### Methods for Preparing Formulations for Stabilizing One or More Component of A PCR Or RT-PCR Reaction Mixture at Ambient Temperatures

Methods for preparing the formulations described herein for substantially stable storage of one or more component of a PCR or RT-PCR reaction mixture at ambient temperatures employ techniques that are well-known to those skilled in the art and generally use commercially available reagents. In some embodiments, the formulations are prepared as concentrated stock solutions of the formulation reagents, e.g., 2×, 5×, 10×, 20× or the like, so as to be admixed with the one or more component of a PCR or RT-PCR reaction mixture at the appropriate ratios to form the desired concentrations.

### Compositions of One or More Stably Stored Component of A PCR or RT-PCR Reaction Mixture at Ambient Temperatures

In some embodiments, compositions are provided of one or more stably stored component of a PCR or RT-PCR reaction mixture at ambient temperatures. In some embodiments, the one or more stably stored component of a PCR or RT-PCR reaction mixture is admixed with a formulation provided herein.

### Articles of Manufacture

In certain embodiments, articles of manufacture are provided, which comprise a formulation provided herein contained in a suitable container or vessel. These articles of manufacture may be used for substantially stable storage of one or more component of a PCR or RT-PCR reaction mixture. In some embodiments, the formulation is selected from those set forth in Table 1. In some embodiments, these articles of manufacture are used for substantially stable storage of one or more component of a PCR or RT-PCR reaction mixture. In some embodiments, these articles of manufacture are used in the kits and methods described herein.

### Kits

In certain aspects of the invention, there are provided kits comprising any one of the articles of manufacture comprising the formulations of the present invention and a package insert. In some embodiments, the components of the kit are supplied in a container, such as a compartmentalized plastic enclosure. In some embodiments, the container has a hermetically sealable cover so that the contents of the kit can be sterilized and sealed for storage prior to use.

### Methods for Substantially Stable Storage of One or More Component of a PCR Or RT-PCR Reaction Mixture at Ambient Temperatures

In another aspect of the present invention, methods are provided for substantially stable storage of one or more component of a PCR or RT-PCR reaction mixture at ambient temperatures.

In certain embodiments, the methods comprise admixing one or more component of a PCR or RT-PCR reaction mixture with a formulation described herein for substantially stable storage of one or more component of a PCR or RT-PCR reaction mixture, wherein the one or more component of a PCR or RT-PCR reaction mixture remains stabilized after storage for a period of at least three months. In some embodiments, at least 95%, at least 90%, at least 85%, at least 80%, at least 75%, at least 70%, at least 65%, at least 60%, at least 55%, at least 50% of the one or more component of a PCR or RT-PCR reaction mixture remains stabilized at ambient temperatures for a period of at least three months. In some embodiments, at least 80% of the one or more component of a PCR or RT-PCR reaction mixture remains stabilized at ambient temperatures for a period of at least three months.

In some embodiments, at least 95%, at least 90%, at least 85%, at least 80%, at least 75%, at least 70%, at least 65%, at least 60%, at least 55%, at least 50% of the one or more component of a PCR or RT-PCR reaction mixture remains stabilized at ambient temperatures for a period of at least one month, at least two months, at least three months, at least four months, at least five months, at least six months, at least seven months, at least eight months, at least nine months, at least ten months, at least 11 months, at least 12 months, at least 15 months, at least 18 months, at least 21 months, at least 24 months. In some embodiments, at least 80% of the one or more component of a PCR or RT-PCR reaction mixture remains stabilized at ambient temperatures for a period of at least one month, at least two months, at least three months, at least four months, at least five months, at least six months, at least seven months, at least eight months, at least nine months, at least ten months, at least 11 months, at least 12 months, at least 15 months, at least 18 months, at least 21 months, at least 24 months.

In some embodiments, the one or more component of a PCR or RT-PCR reaction mixture comprise a PCR master mix or a RT-PCR master mix. In some embodiments, the one or more component of a PCR or RT-PCR reaction comprise a RT-PCR internal control template nucleic acid. In some embodiments, the one or more component of a PCR or RT-PCR reaction mixture comprise an armored RNA. In some embodiments, the one or more component of a PCR or RT-PCR reaction mixture comprise a polymerase. In some embodiments, the one or more component of a PCR or RT-PCR reaction mixture comprise a DNA polymerase. In some embodiments, the one or more component of a PCR or RT-PCR reaction mixture comprise an armored RNA and a polymerase.

In certain embodiments, the formulation is one of the formulations set forth in Table 1.

The methods as described herein may use the articles of manufacture and kits disclosed herein.

The following Examples are presented by way of illustration and not limitation.

### EXAMPLE 1

### Stabilization of aRNA for at Least One Month at Above Ambient Temperatures

This Example describes formulations and compositions of the present invention for stabilizing the Quantification Standard (QS)-armored RNA (aRNA).

A volume of 75 μl of the aRNA stock solution (Roche, of similar composition to HIV-1 QS as part of kit #03542998 190 comprising Tris-HCl, EDTA, armored HIV-1 RNA, and sodium azide) was combined with 75 μl of stabilizer and incubated at the noted temperature.

At the noted time points, aRNA samples were processed either by heat-rupture or using a MagNA Pure compact total nucleic acid isolation kit.

For heat-rupture, 2 μl of aRNA sample was added to 18 μl of elution buffer (Roche, of similar composition to EB as part of kit #03542998 190 comprising Tris and methylparaben). This was then heated to 70° C. for 5 minutes, and immediately placed on ice. A volume of 4 μl of this was used as template in an 35 μl final volume RT-PCR reaction with the following other components: 12.5 μl of elution buffer containing approximately 1500 copies of an HIV target RNA, 7 μl of Mn²⁺ solution (Roche, of similar composition to CAP/CMT Mn²⁺ as part of kit #03542998 190 comprising manganese acetate, sodium azide, acetic acid), 1 μl of BSA solution (50 mg/ml in elution buffer), and 10.5 μl of RT-PCR mastermix solution (Roche, of similar composition to HIV-1 MMX as part of kit #03542998 190, but using a modified polymerase, and comprising tricine, potassium acetate, potassium hydroxide, glycerol, dimethylsulfoxide, sodium azide, dATP, dCTP, dGTP, dUTP, uracil N-glycosylase, aptamer, primer mixture, probe mixture, pH 8.1).

For MagNA Pure processing, 10 μl of aRNA sample was added to 190 μl of human plasma containing approximately 5000 copies of an HIV RNA and processed using the Total Nucleic Acid Isolation Kit as per the manufacturer's instructions. The elution volume was 100 μl. A volume of 16.5 μl of the elution was used as template in a 35 μl final volume RT-PCR reaction with the following other components: 7 μl of Mn²⁺ solution, 1 μl of BSA solution, and 10.5 μl of RT-PCR mastermix solution.

RT-PCR reactions were performed on a BioRad CFX96, with the following cycling conditions: 55° C. for 30 seconds, 60° C. for 30 seconds, 65° C. for 30 seconds, 5 cycles of 95° C. for 3 seconds and 55° C. for 15 seconds followed by a plate read, and 38 cycles of 91° C. for 5 seconds and 58° C. for 15 seconds followed by a plate read.

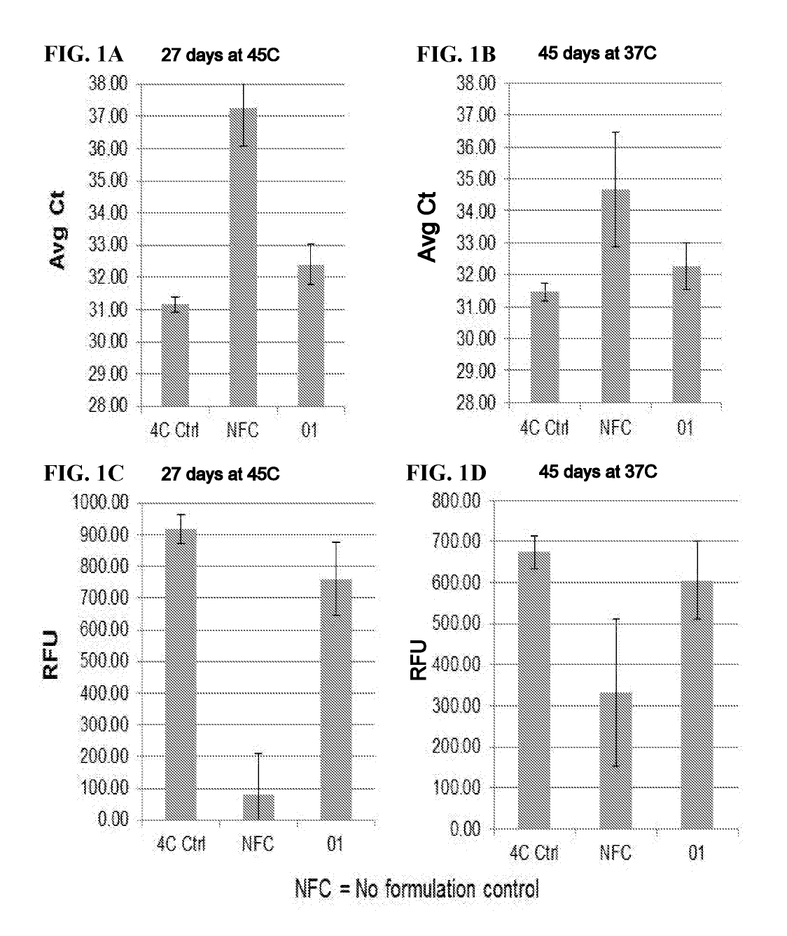

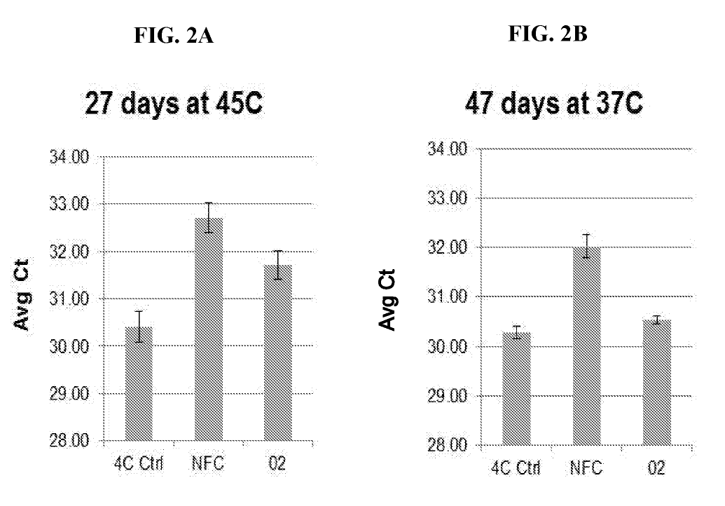

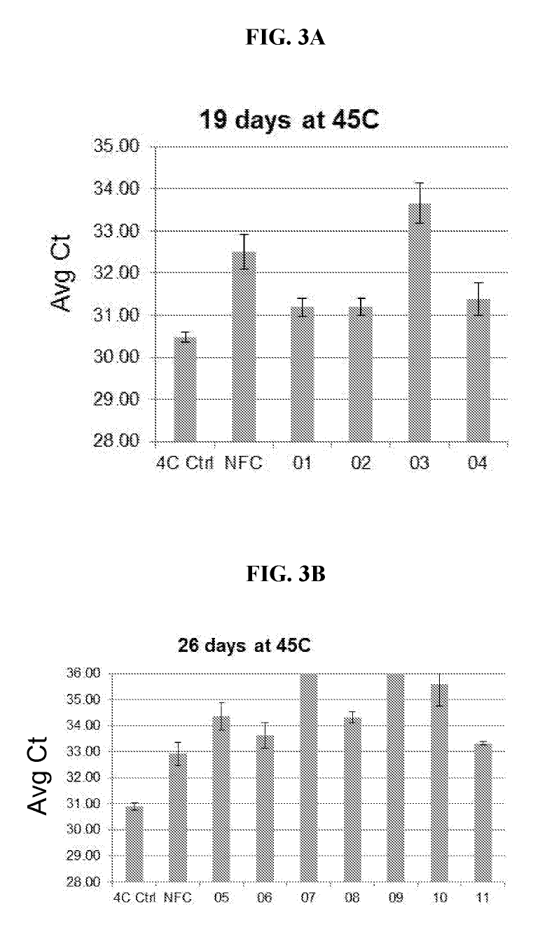

Exemplary formulations are shown in Table 2 below. Data in FIGS. 1A-D shows that the samples containing formulation 01 had lower Ct values and improved fluorescence compared to the NFC, indicating an increased amount of RNA yield from those samples. FIGS. 2A-B show that the samples containing formulation 02 had lower Ct values compared to the NFC, indicating an increased amount of RNA yield from those samples. FIGS. 3A-B show that the samples containing formulations 01, 02, and 04 had lower Ct values compared to the NFC, indicating an increased amount of RNA yield from those samples. All other formulations tested in these experiments showed Ct values at or above those for the NFC, indicating no improved RNA yield or stability (FIGS. 3A-B).

TABLE 2
Exemplary Storage Formulations

| Name | Component 1 | Component 2 | Component 3 |
|---|---|---|---|
| 01 | 10% Lactic Acid | 50 mM Boric Acid | 10 mM Sodium |
| | | | Tetraborate |
| 02 | 7.5% Lactic Acid | 324 mM Boric Acid | |
| 03 | | 50 mM Boric Acid | 10 mM Sodium |
| | | | Tetraborate |
| 04 | 10% Lactic Acid | | |
| 05 | 10% Malic Acid | 100 mM Boric Acid | |
| 06 | 2% Malic Acid | 20 mM Boric Acid | |
| 07 | 10% Malonic Acid | 100 mM Boric Acid | |
| 08 | 2% Malonic Acid | 20 mM Boric Acid | |
| 09 | 10% Tartaric Acid | 100 mM Boric Acid | |
| 10 | 2% Tartaric Acid | 20 mM Boric Acid | |
| 11 | 2% Citric Acid | 20 mM Boric Acid | |

### EXAMPLE 2

### Stabilization of RT-PCR Mastermix

This Example describes formulations and compositions of the present invention for stabilizing RT-PCR mastermix (Roche, of similar composition to HIV-1 MMX as part of kit #03542998 190) for a period of at least 14 days at elevated temperatures.

A volume of 84 μl of stabilizer was combined with 84 μl of the 1.5× RT-PCR mastermix and stored at the indicated temperatures. At each time point, 14 μl of the RT-PCR mastermix-stabilizer mixture was added to a 35 μl final volume RT-PCR reaction with the following other components: 3.5 μl of a Mn²⁺ solution, 1 μl of BSA solution, and 16.5 μl of elution buffer containing approximately 1×10⁴ copies of purified QS RNA target and 100 copies of purified HIV RNA target. RT-PCR reactions were performed on a BioRad CFX96, with the following cycling conditions: 55° C. for 30 seconds, 60° C. for 30 seconds, 65° C. for 30 seconds, 5 cycles of 95° C. for 3 seconds and 55° C. for 15 seconds followed by a plate read, and 38 cycles of 91° C. for 5 seconds and 58° C. for 15 seconds followed by a plate read.

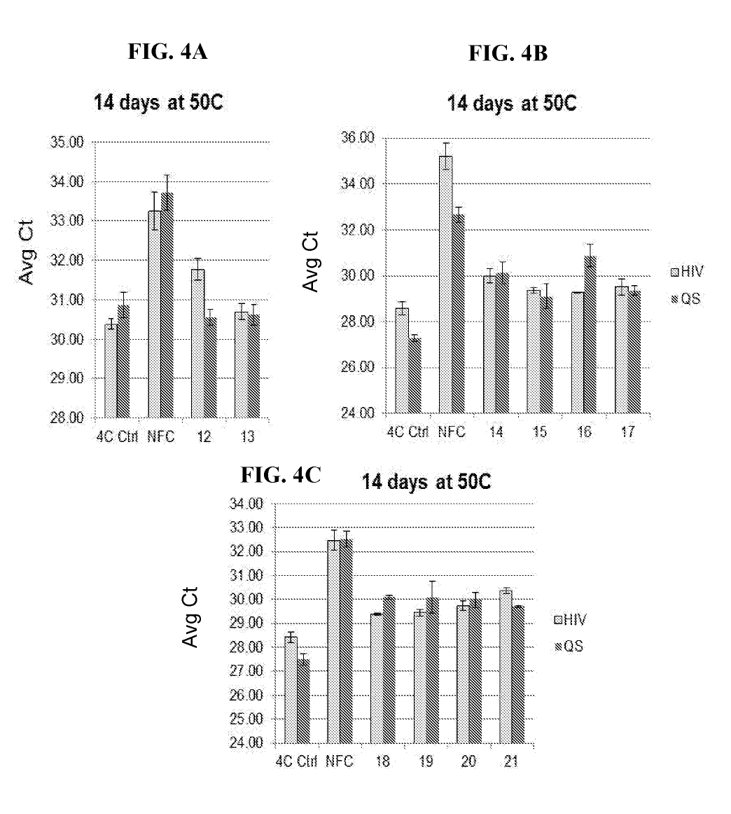

Exemplary formulations are shown in Table 3 below. Data in FIGS. 4A-C shows that the samples containing indicated formulations had lower Ct values for both RT-PCR targets (HIV and QS) compared to the NFC.

TABLE 3
Exemplary Storage Formulations

| Name | Component 1 | Component 2 |
|---|---|---|
| 12 | 50% Sucrose | 50 mM Tris-HCl pH 8.0 |
| 13 | 900 mM Sucrose | 50 mM Taurine pH 8.8 |
| 14 | 5% (v/v) Dimethylpropionamide | 50 mM Taurine pH 8.8 |
| 15 | 5% (v/v) Dimethylacetamide | 50 mM Taurine pH 8.8 |
| 16 | 10% (w/v) PEG 8000 | 50 mM Taurine pH 8.8 |
| 17 | 10% (w/v) PEG 400 | 50 mM Taurine pH 8.8 |
| 18 | 5% (v/v) Dimethylformamide | 100 mM Taurine pH 8.8 |
| 19 | 5% (v/v) Butyramide | 100 mM Taurine pH 8.8 |
| 20 | 5% (v/v) Dimethylisobutyramide | 100 mM Taurine pH 8.8 |
| 21 | 5% (v/v) Methylpropionamide | 100 mM Taurine pH 8.8 |

Unless the context requires otherwise, throughout the present specification and claims, the word “comprise” and variations thereof, such as, “comprises” and “comprising,” which is used interchangeably with “including,” “containing,” or “characterized by,” is inclusive or open-ended language and does not exclude additional, unrecited elements or method steps. The phrase “consisting of” excludes any element, step, or ingredient not specified in the claim. The phrase “consisting essentially of” limits the scope of a claim to the specified materials or steps and those that do not materially affect the basic and novel characteristics of the claimed invention. The present disclosure contemplates embodiments of the invention compositions and methods corresponding to the scope of each of these phrases. Thus, a composition or method comprising recited elements or steps contemplates particular embodiments in which the composition or method consists essentially of or consists of those elements or steps.

Reference throughout this specification to “one embodiment” or “an embodiment” or “an aspect” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The various embodiments described above can be combined to provide further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. An admixture comprising:
(a) a formulation for substantially stable storage of one or more component of a PCR or RT-PCR reaction mixture at ambient temperatures, comprising:
(i) a hydroxyacid, and
(ii) a borate composition; and
(b) one or more component of a PCR or RT-PCR reaction mixture;
wherein the one or more component of the PCR or RT-PCR reaction mixture remains stabilized after storage at ambient temperatures for a period of at least three months.

2. The admixture of claim 1, wherein the hydroxyacid is a compound of formula (I): 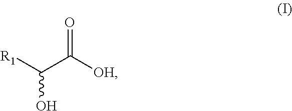
wherein R1 is selected from H, unsubstituted or substituted alkyl, or unsubstituted or substituted aryl.

3. The admixture of any one of claims 1-2, wherein the hydroxyacid is selected from the group consisting of lactic acid, malic acid, malonic acid, tartaric acid, citric acid, and combinations thereof.

4. The admixture of any one of claims 1-3, wherein the hydroxyacid is lactic acid.

5. The admixture of any one of claims 1-4, wherein the borate composition comprises a compound selected from the group consisting of boric acid, borate, sodium tetraborate, borax, and a combination thereof.

6. The admixture of any one of claims 1-5, wherein the formulation comprises (i) lactic acid and boric acid; or (ii) lactic acid, boric acid, and sodium tetraborate.

7. The admixture of any one of claims 1-6, wherein the one or more component of a PCR or RT-PCR reaction mixture comprises a control nucleic acid.

8. The admixture of any one of claims 1-6, wherein the one or more component of a PCR or RT-PCR reaction mixture comprises an armored RNA.

9. An admixture comprising:
(a) a formulation for substantially stable storage of one or more component of a PCR or RT-PCR reaction mixture at ambient temperatures, comprising:
(i) a non-reducing sugar, and
(ii) a buffer; and
(b) one or more component of a PCR or RT-PCR reaction mixture;
wherein the one or more component of the PCR or RT-PCR reaction mixture remains stabilized after storage at ambient temperatures for at least three months.

10. The admixture of claim 9, wherein the non-reducing sugar is selected from the group consisting of sucrose and trehalose.

11. The admixture of any one of claims 9-10, wherein the buffer is selected from the group consisting of Tris-HCl, citric acid, tartaric acid, malic acid, sulfosalicylic acid, sulfoisophthalic acid, oxalic acid, borate, CAPS (3-(cyclohexylamino)-1-propanesulfonic acid), CAPSO (3-(cyclohexylamino)-2-hydroxy-1-propanesulfonic acid), EPPS (4-(2-hydroxyethyl)-1-piperazinepropanesulfonic acid), HEPES (4-(2-hydroxyethyl)piperazine-1-ethanesulfonic acid), MES (2-(N-morpholino)ethanesulfonic acid), MOPS (3-(N-morpholino)propanesulfonic acid), MOPSO (3-morpholino-2-hydroxypropanesulfonic acid), PIPES (1,4-piperazinediethanesulfonic acid), TAPS (N-[tris(hydroxymethyl)methyl]-3-aminopropanesulfonic acid), TAPSO (2-hydroxy-3-[tris(hydroxymethyl)methylamino]-1-propanesulfonic acid), TES (N-[tris(hydroxymethyl)methyl]-2-aminoethanesulfonic acid), bicine (N,N-bis(2-hydroxyethyl)glycine), tricine (N-[tris(hydroxymethyl)methyl]glycine), tris (tris(hydroxymethyl)aminomethane), and bis-tris (2-[bis(2-hydroxyethyl)amino]-2-(hydroxymethyl)-1,3-propanediol).

12. The admixture of any one of claims 9-11, wherein the formulation comprises sucrose and Tris-HCl.

13. An admixture comprising:
(a) a formulation for substantially stable storage of one or more component of a PCR or RT-PCR reaction mixture at ambient temperatures, comprising:
(i) an aminosulfonic acid or ammonium sulfate, and
(ii) at least one of a non-reducing sugar, an amide, or polyethylene glycol; and
(b) one or more component of a PCR or RT-PCR reaction mixture;
wherein the one or more component of thePCR or RT-PCR reaction mixture remains stabilized after storage at ambient temperatures for a period of at least three months.

14. The admixture of claim 13, wherein the formulation comprises an aminosulfonic acid of formula (II): 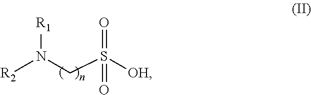
wherein R1 and R2 are independently selected from H, unsubstituted or substituted alkyl, and unsubstituted or substituted aryl.

15. The admixture of any one of claims 13-14, wherein the aminosulfonic acid is taurine.

16. The admixture of claim 13, wherein the formulation comprises ammonium sulfate.

17. The admixture of any one of claims 13-16, wherein the formulation comprises a non-reducing sugar selected from the group consisting of sucrose and trehalose.

18. The admixture of any one of claims 13-17, wherein the non-reducing sugar is sucrose.

19. The admixture of any one of claims 13-18, wherein the formulation comprises an amide selected from the group consisting of a compound of formula (III): 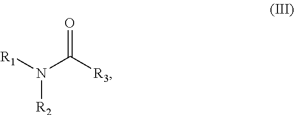
wherein R1, R2, and R3 are independently selected from H, unsubstituted or substituted alkyl, and unsubstituted or substituted aryl.

20. The admixture of any one of claims 13-18, wherein the formulation comprises an amide selected from the group consisting of N,N-dimethylpropionamide, N,N-dimethylacetamide, butyramide, and dimethylformamide.

21. The admixture of any one of claims 13-18, wherein the formulation comprises dimethylisobutyramide.

22. The admixture of any one of claims 13-18, wherein the formulation comprises methylpropionamide.

23. The admixture of any one of claims 13-18, wherein the formulation comprises polyethylene glycol.

24. The admixture of claim 23, wherein the polyethylene glycol is PEG 8000 or PEG 400.

25. The admixture of any one of claims 1-24, wherein the one or more component of a PCR or RT-PCR reaction mixture comprises a polymerase.

26. A method for substantially stable storage of one or more component of a PCR or RT-PCR reaction mixture at ambient temperatures, comprising: admixing one or more component of a PCR or RT-PCR reaction mixture with a formulation selected from the group consisting of:
a) a formulation comprising a hydroxyacid, and a borate composition;
b) a formulation comprising a non-reducing sugar and a buffer
c) a formulation comprising i) an aminosulfonic acid or ammonium sulfate, and ii)
at least one of a non-reducing sugar, an amide, or polyethylene glycol;
wherein the one or more component of a PCR or RT-PCR reaction mixture remains stabilized after storage at ambient temperatures for a period of at least three months.

27. The method of claim 26, wherein at least 80% of the one or more component of a PCR or RT-PCR reaction mixture remains stabilized at ambient temperatures for a period of at least three months.

28. The method of any one of claims 26-27, wherein the one or more component of a PCR or RT-PCR reaction mixture is a RT-PCR master mix, a RT-PCR internal control template nucleic acid, or a combination thereof.
